(19) United States
(12) Patent Application Publication
(10) Pub. No.: US 2011/0207123 A1
(43) Pub. Date: Aug. 25, 2011

(54) METHOD FOR DETECTING CHROMOSOMAL ABNORMALITIES

(76) Inventors: Pierrick Serge Romana, Deuil La Barre (FR); Marc Le Lorc'h, Saint remy les chevreuses (FR); Héra Der-Sarkissian, Le Perreux sur marne (FR)

(21) Appl. No.: 12/737,551

(22) PCT Filed: Jul. 24, 2009
(86) PCT No.: PCT/FR2009/051499

§ 371 Date: Apr. 18, 2011

(30) Foreign Application Priority Data
Jul. 25, 2008 (FR) 0855114

(51) Int. Cl. C12Q 1/68

(52) U.S. Cl. 435/6.11

(57) ABSTRACT

The invention relates to an in vitro method for detecting chromosomal abnormalities in a mammal, comprising the in situ hybridization of n chromosomes from a metaphase chromosome preparation with sets of a plurality of nucleic acids, each set hybridizing, over a length of 700 000 to 3 000 000 contiguous bp, specifically to the subtelomeric ends specific to said chromosomes, each set being detectably labeled with a fluorochrome, such that each chromosome is distinguishable by a particular fluorochrome or a particular fluorochrome combination.

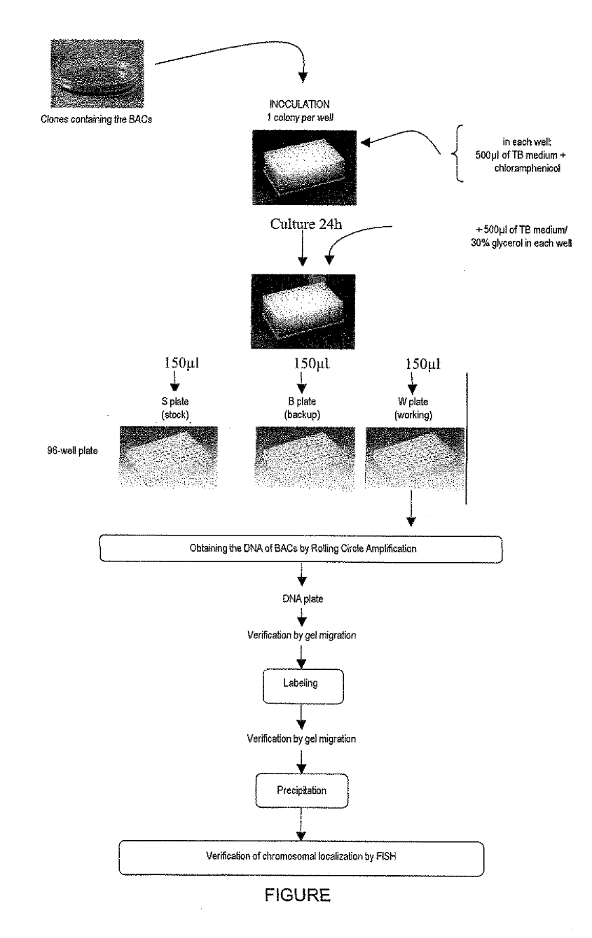

The invention relates to the detection of chromosomal abnormalities by hybridization of specific nucleic acid probes.

### TECHNOLOGICAL BACKGROUND

Chromosomal abnormalities, whether they are constitutional or acquired, are responsible for many pathological conditions. They may be balanced (i.e. not accompanied by a loss of chromosomal material) or unbalanced, i.e. accompanied by losses of or gains in chromosomal material. The term then used is genomic imbalances. With the techniques that are routinely available today (band karyotype and fluorescent in situ hybridization), it is estimated that chromosomal abnormalities are responsible for 10 to 15% of instances of mental retardation and of congenital malformations. Moreover, they are very frequently found associated in cancer cells. In a very large number of cases, they determine the prognosis thereof, and they make it possible to understand the molecular cause thereof and to envision targeted treatments. This is why, whether it is in constitutional pathology or in cancers, the diagnosis of chromosomal abnormalities is fundamental, both for treatment and for research.

Chromosomal abnormalities may or may not be visible under the microscope. When they are visible, they are detected by conventional karyotyping techniques. Developed in the 1960s, these techniques make it possible to visualize the chromosomes with a succession, along their longitudinal axis, of light and darker bands characteristic of each pair of chromosomes. Examination of the overall morphology of the chromosomes and of the organization of the various bands makes it possible to establish the karyotype. The smallest of the bands that can be observed is 5 million base pairs (5 megabases or Mb). This is a first limit to this technique. A second is that exchanges of telomeric regions, of the same size and the shade of which (generally black) is identical, cannot be detected by conventional karyotyping techniques. These abnormalities that cannot be detected by band techniques are referred to as cryptic.

One approach for detecting these cryptic chromosomal abnormalities uses specific fluorescent probes which hybridize to the chromosomes. This technique, called FISH for “fluorescence in situ hybridization” (FISH), consists in hybridizing, to the target DNA, a complementary DNA fragment into which a fluorochrome has been introduced (operation referred to as labeling). Such a DNA fragment is called a probe. Fluorescence in situ hybridization can be carried out on metaphase chromosomes, in which case it uses probes specific for a chromosome, for an arm, for a chromosomal region or for a given locus. The chromosomal abnormalities are revealed by visualization, under a microscope, of the fluorescent sites corresponding to the sites of the target DNA that have hybridized with the probes.

One of the major indications for FISH is the detection of cryptic telomeric abnormalities. These abnormalities occasion by themselves close to 5% of instances of mental retardation with malformations. Moreover, in cancers, telomeric abnormalities (balanced in these pathological conditions) of which the frequency is high have been described. Thus, t(12; 21) in childhood acute leukemia is invisible by conventional cytogenetic techniques, although it is the most frequent genetic abnormality in this pathological condition (Romana et al, Genes Chromosomes Cancer, 1994, 9(3): 186-91). The systems for detecting chromosomal abnormalities that are for the moment commercially available are composed of one or two probes representing the telomeric ends of one or two chromosomal pairs. This supposes, if it is desired to study all the ends, hybridizing these probes on one or more slides, depositing them on various parts of these slides. This system supposes chromosomal preparations rich in metaphases in order for mitoses to be present on all the parts of the slide where the various probes are deposited. These prerequisites cannot be achieved for preparations of chromosomes which come from amniocyte cultures in prenatal constitutional cytogenetics, nor in cancers. For these two situations, it remains necessary to develop a system making it possible to study all the chromosomal ends on one or two mitoses.

This is the problem that Brown et al (Nature Medicine, 7(4): 497-501) wanted to address in developing the M-Tel system. In this system, the telomeric probes for a chromosome are labeled with a specific fluorochrome combination. Thus, they were able to produce probes specific for 12 chromosomes, each detectable after hybridization on a single metaphase. Using two batches of probes, all of the 23 chromosomal pairs could be explored by studying two mitoses. This test, subsequently used in the study Brown et al, 2002, Blood, 99(7): 2526-2531, nevertheless has certain drawbacks. In particular, the probes used produce a weak signal, with a signal/noise ratio which is much too high.

There still existed a need for a test for detecting cryptic telomeric abnormalities which is rapid, practical and reliable. The test proposed by the inventors meets these requirements and can also be used not only for the diagnosis of prenatal and postnatal constitutional chromosomal pathological conditions, but also for the diagnosis of those associated with cancers. This test is also particularly suitable for the detection of balanced chromosomal abnormalities.

### SUMMARY OF THE INVENTION

The invention relates to an in vitro method for detecting chromosomal abnormalities in an animal, preferably a vertebra, more preferably a mammal, comprising:
the in situ hybridization of n chromosomes from a metaphase chromosome preparation with sets of a plurality of nucleic acids, each set hybridizing, over a length of 700 000 to 3 000 000 bp, specifically to the subtelomeric ends specific to said chromosomes, each set being detectably labeled with a fluorochrome, such that each chromosome is distinguishable by a particular fluorochrome or a particular fluorochrome combination,
   n being an integer between 2 and the total number of chromosomes of said animal,
   the total number of fluorochromes used being an integer less than n,
the detection of the fluorescence emitted by the fluorochromes, whereby chromosomal abnormalities are detected.

### FIGURE LEGEND

The FIGURE shows a general protocol for obtaining the DNA of telomeric clones.

### DETAILED DESCRIPTION OF THE INVENTION

### Definitions

A “specific subtelomeric end” is the specific region of a chromosome which is closest to the telomeric consensus sequence of a chromosome (common to all chromosomes). It is generally located between 100 and 300 kb from the consensus sequences.

A “telomere” or “telomeric end” is a highly repetitive, noncoding region of DNA at the end of a chromosome. In humans, the TTAGGG sequence is telomere-specific and is repeated several hundred times. This sequence is common to all chromosomes.

“Specific hybridization” denotes the ability of a set of a plurality of nucleotides to hybridize to a given DNA sequence. In other words, a set which hybridizes specifically to a subtelomeric end of a chromosome does not hybridize to another subtelomeric end. For example, a set which hybridizes specifically to the subtelomeric end of the long arm of chromosome 1 will not hybridize to the subtelomeric end of the short arm of chromosome 1, nor to any other sequence of this chromosome or of another chromosome.

For reasons of convenience, the chromosome suspected of being affected by an abnormality is, in the subsequence text, referred to as “target chromosome”.

### Sample Preparation

The test samples are preparations of biological fluids or tissues containing metaphase chromosomes from an animal. They are preferably blood samples, amniotic fluid samples in the case of prenatal diagnoses or bone marrow samples for malignant hemopathies.

The animal is preferably a mammal, preferably a human being, regardless of the sex or age thereof. It may be an embryo, a fetus, a newborn, a child, an adolescent or an adult.

Samples are prepared as for a conventional cytogenetic analysis on slides, whether it is a question of samples placed in culture or analyzed directly.

A general procedure for preparing metaphase chromosomes consists in, if possible, culturing the cells containing the chromosomes to be analyzed and in arresting them in mitosis with a mitotic spindle inhibitor, for example with colchicine. The chromosomes are then fixed with an acetic acid/methanol mixture and then spread on a slide.

### Probes Used:

The method of the invention uses nucleic acid probes which cover a length of 700 000 to 3 000 000 bases (bp) contiguous of at least one subtelomeric end of each target chromosome. Preferably, the probes cover at least 800 000 bp, preferably at least 1 Mb, more preferably 1 to 1.5 Mb, of the chromosome-specific subtelomeric ends.

The probes are constituted of a set of a plurality of nucleic acids (hereinafter “a set”) covering all or parts of the subtelomeric end specific for the long arm or for the short arm of the target chromosome. The plurality of nucleic acids is composed of nucleic acids complementary to contiguous parts of the subtelomeric end for which the set is specific. These contiguous parts may optionally be slightly overlapping.

The subtelomeric end to which the probes hybridize is not that of a short arm of an acrocentric chromosome (i.e. chromosomes 13, 14, 15, 21 and 22 in human beings).

As illustrated in table 2, the sets of nucleic acids hybridize at a varying distance from the telomere, according to the chromosome targeted. For example, the sets of nucleic acids used in the examples hybridize at a distance of 4110 bp from the proximal end of telomere 21q, or 828 698 bp from the proximal end of telomere 1p. This distance is related to the probe specificity imperative. Specifically, some regions, even subtelomeric regions, of a given chromosome can have DNA sequences in common with other chromosomes. The size of these shared sequences can be variable according to the chromosomes. This explains the variability, according to chromosomes, in the distance that exists between the telomeric consensus sequence and the distal end of the specific subtelomeric probe.

The total number of chromosomes present in the normal cells of a given animal is fixed and known. Thus, the cells of a human being normally comprise 23 pairs of chromosomes, one pair of which corresponds to the XX or XY chromosomes. In the method of the invention, n chromosomes are simultaneously analyzed, n being an integer between 2 and the total number of chromosomes of the animal. Preferably, at least 12 chromosomes are simultaneously analyzed.

According to one preferred embodiment of the invention, the set of a plurality of nucleic acids is produced by amplification of one or more BACs into which a fragment of the subtelomeric end of the target chromosome is inserted. Various internet sites make it possible to visualize the position of these BACs along each chromosome, for example, on the UCSC site (http://genome.ucsc.edu/).

The amplification can be carried out by any means of DNA amplification known to those skilled in the art. By way of example, mention may be made of PCR amplification or rolling circle amplification (RCA).

### Probe Labeling:

In the method of the invention, each set is detectably labeled with a fluorochrome, such that each chromosome is distinguishable from the other chromosomes by a particular fluorochrome or a particular fluorochrome combination.

The nucleic acids making up the set of a plurality of nucleic acids can be labeled with nucleotides conjugated with a fluorescent molecule, or fluorochrome (direct labeling).

Incorporation of nucleotides conjugated with a fluorochrome into the probes of the invention can be carried out by any means known to those skilled in the art, in particular by nick-translation or PCR.

Alternatively, the nucleic acids can be labeled with nucleotides conjugated with a molecule that is specifically recognized by a ligand, preferably an antibody conjugated to a fluorochrome (indirect labeling).

More particularly, the indirect labeling of the probes used for the in situ hybridization of the target chromosome is carried out by coupling each of them to a hapten, and by means of an affinity reaction between this hapten and a ligand capable of specifically binding to said hapten and which is either conjugated to a fluorochrome, or made fluorescent by reaction with a counterligand conjugated to a fluorochrome.

By way of examples of haptens that can be used for indirect labeling of the probes used in the present invention, mention may in particular be made of:
digoxigenin, dinitrophenol and 2-acetylaminofluorene, in which case the hapten/ligand affinity reaction is advantageously carried out by means of an antibody directed against these compounds and conjugated to a fluorochrome;
biotin, in which case the hapten/ligand affinity reaction is advantageously carried out by means of avidin conjugated to a fluorochrome, or by using anti-biotin antibodies, and then antibodies directed against these antibodies and which are conjugated to a fluorochrome.

The fluorochromes used can be chosen, without distinction, from various fluorescent compounds used for labeling nucleic probes, such as fluorescein (sodium fluoresceinate) and derivatives thereof such as fluorescein isothiocyanate (FITC); rhodamine and derivatives thereof, such as tetramethyl rhodamine isothiocyanate (TRITC); diamidino-phenylindole (DAPI); acridine; fluorescent dyes comprising reactive amines, such as 6-((7-amino-4-methylcoumnarin-3-acetyl)amino)hexanoic acid succinimidyl ester (A. NICA); the fluorescent dyes sold under the trade names BODIPY, such as BODIPY′J, FR-Br, BODIPY R6G, BODIPY TMR, BODIPY TR and BODIPY 530/550 sold by the company Bio-Rad Inc. (USA), the dyes Cascade Blue (Trilink BioTechnologies USA), Cy2, Cy3, Cy3.5, CYS, Cy5.5, and Cy7 (Bio-Rad Inc., USA), DABCYL and EDANS (Eurogentec, BE); eosin; erythrosine; 6-Farn and Texas Red. Coumarin and its derivative diethyl aminomethyl coumarin (DEAC) are fluorochromes that are also preferred.

The method according to the invention is a multifluorescence method. In other words, the number of fluorochromes used for detecting chromosomal abnormalities is less than the number n of chromosomes analyzed. The chromosomes are therefore combinatorially labeled so as to be able to distinguish them from one another. This chromosome labeling is carried out by each set of a plurality of nucleic acids.

Preferably, the subtelomeric ends of the short or long arms (p and q, respectively) of the same chromosome are labeled with the same fluorochrome or the same fluorochrome combination.

In one preferred embodiment, at least four fluorochromes are used (for example, a direct labeling with the fluorochromes FITC, rhodamine and coumarin, and an indirect labeling with biotin, the latter being revealed with Cy5-labeled streptavidin), which allows 15 different combinations of fluorochromes (2⁴-1). In order to detect abnormalities on all the chromosomes of an animal having more than 15 chromosomes, it is then possible to use two batches of probes (for 12 chromosomes for one and 11 chromosomes for the other for the study of human chromosomes, for a total of 41 probes which hybridize all the ends of the long and short arms of human chromosomes, except the short arms of the acrocentric chromosomes). Thus, all the telomeric ends can be studied on the basis of the analysis of one mitosis selected on two different areas of a slide on which the two batches of probes have been deposited. Preferably, the batches of probes are grouped together so as to avoid analyzing, in the same group, chromosomes which resemble one another from a cytogenetic point of view (for example, human chromosomes 21 and 22 are preferably analyzed by 2 different batches).

Table 1 below illustrates theoretically the 15 possible combinations for 4 different fluorochromes:

TABLE 1

| Labeling | FITC | Rhodamine | Coumarin | Biotin |
|---|---|---|---|---|
| 1 | X | | | |
| 2 | | X | | |
| 3 | | | X | |
| 4 | | | | X |
| 5 | X | X | | |
| 6 | X | | X | |
| 7 | X | | | X |
| 8 | X | X | X | |
| 9 | X | X | | X |
| 10 | X | | X | X |
| 11 | | X | | |
| 12 | | X | X | |
| 13 | | X | | X |
| 14 | | | X | |
| 15 | | | X | X |

### Hybridization:

The hybridization is carried out according to conventional in situ hybridization conditions (Romana et al. 1994, above). By way of indication, the metaphase chromosomes in suspension in an acetic acid//methanol mixture (1/3-2/3) are spread on slides. After having been air-dried, they are amassed in a 2×SSC saline solution for 5 minutes, dehydrated in solutions of increasing alcohol concentration (60% to 100%) and air-dried.

The two probe batches are then denatured for 10 minutes at 70° C. in 50% formamide (FLUKA)/2×SSC (Standard Sodium Citrate) pH 7. The denatured probes are deposited on the slide at two positions and covered with two cover slips hermetically sealed with a rubber cement adhesive. The whole assembly is placed on a hotplate for 3 to 4 minutes. The whole assembly is then hybridized for 18 hours at 37° C. in a humid chamber. The post-hybridization washes are carried out at 72° C. for 5 minutes in 1×SSC buffer.

In the case of the use of probes labeled with digoxigenin or biotin in particular, the probes are detected using antibodies coupled to different fluorochromes.

In the case of the use of probes directly labeled with fluorochromes, counterstaining of the chromosomes with DAPI (1 μg/ml, Molecular Probes) and mounting of the slides in p-phenyldiamine can immediately be carried out.

Variations of these procedures are given in the experimental section.

### Visualization:

Revealing is generally carried out after 18 hours of incubation at 37° C.

The slides are observed using an epifluorescence microscope equipped with a CCD camera and appropriate filters. The images are analyzed using an image-processing computer system.

A chromosomal abnormality will be detected in the event of an absence of signal at the level of a subtelomeric end of a target chromosome, or in the event of a signal which is not located on the chromosome corresponding to the probe.

### Applications:

The method of the invention can be used for detecting two chromosomes or more, or preferably for all the chromosomes of the chromosome preparation.

The method of the invention can be used not only for the diagnosis of constitutional chromosomal pathological conditions, but also for cancer diagnosis. This test is also particularly suitable for detecting balanced chromosomal abnormalities.

It is in particular useful in routine diagnosis, for verifying chromosomal abnormalities suspected due to a chromosomal examination by band techniques.

Moreover, it can be used for the diagnosis of cryptic chromosomal abnormalities being prenatal and prenatal constitutional chromosomal pathology, and in cancers, in particular in malignant hemopathies (where they are often balanced).

The region of hybridization between the subtelomeric end analyzed and the set of a plurality of nucleic acids which is specific therefor allows the detection of a signal of strong intensity. The main advantage of this is that it increases the signal/noise ratio.

In addition, the combinatorial fluorescent labeling is particularly advantageous if the amount of samples that is available for the diagnosis is limited. Specifically, as indicated above, the use of a combination of 4 fluorochromes allows an analysis of two mitoses.

The examples and FIGURES illustrate the invention without limiting the scope thereof.

### EXAMPLES

### Example 1

### Construction of 41 telomeric probes

The probes are prepared from BACs (Bacterial Artificial Chromosomes). A BAC is a fragment of human DNA, generally between 150 000 and 200 000 base pairs (150 kb-200 kb) in size, inserted into a plasmid transfected into E. coli.

Various internet sites allow the position of these BACs along each chromosome to be visualized. By consulting the UCSC site (http://genome.ucsc.edu/), the inventors selected 286 BACs located in the subtelomeric regions specific to the long and short arms of all the chromosomes, with the exception of those of the short arms of the 5 acrocentric chromosomes (13, 14, 15, 21 and 22) which are composed of repeat sequences common to these 5 chromosomes. For the 41 ends, (23 chromosomal pairs minus 5), the BACs were selected in a contiguous state over a distance of at least 800 000 base pairs (800 kb) so as to obtain a probe generating a powerful signal.

TABLE 2
Characteristics of the probes used (telo dist = telomere distance)

| Telomeres | Contiguous clones | Position (bp) | Length (bp) | Accession No. | Marker | Genes |
|---|---|---|---|---|---|---|
| 1p | | | | | | |
| | CTD-3113J13 | 828698-1021785 | 193088 | AQ780886 | D1S118 | AGRN |
| | CTD-2587K16 | 1051546-1205883 | 154338 | AQ470435 | SHGC-142119 | SDF4 |
| | RP11-421C4 | 1247484-1432829 | 185345 | BZ774583 | D1S1287 | DVL1 |
| Size: 1 663 320 bp | RP11-846C3 | 1457626-1687885 | 230259 | BX663510 | RH25018 | CDC2L2 |
| | RP11-798H13 | 1734771-1923265 | 188494 | AQ492839 | D1S930 | GNB1 |
| Telo dist: 828 698 bp | RP11-547D24 | 1881315-2014198 | 132883 | AL391845 | D1S1328 | GABRD |
| | RP11-181G12 | 2062347-2242269 | 179922 | AL590822 | RH103396 | SKI |
| | RP11-1012C20 | 2291750-2492018 | 200268 | AQ670062 | RH46873 | PEX10 |
| 1q | | | | | | |
| Size: 1 127 322 bp | RP11-438F14 | 246754133-246932000 | 177867 | AC098483 | D1S554 | OR2T29 |
| | CTD-3000C19 | 246529431-246722177 | 192746 | AQ105134 | D1S3148 | |
| Telo dist: 495 586 bp | RP11-469H22 | 246250426-246455373 | 204947 | AQ630268 | STSG32593FS | |
| | RP11-908P10 | 245985930-246193139 | 207209 | AQ670208 | RH78729 | OR2W3 |
| | CTD-2548B21 | 244064096-244262971 | 198875 | AQ390121 | SHGC-84075 | |
| 2p | | | | | | |
| | RP11-356M6 | 178830-334228 | 155398 | AC079779 | RH103149 | ACP1 |
| Size: 936 223 bp | RP11-1105H20 | 292902-510007 | 217105 | AQ685436 | D2S2147 | |
| | RP11-478N3 | 510170-688604 | 178434 | AQ635663 | D2S1318 | TMEM18 |
| Telo dist: 178 830 bp | RP11-100K14 | 714657-885732 | 171075 | AQ342063 | RH92499 | |
| | CTD-2504K9 | 882429-1115053 | 232624 | AQ265200 | RH99251 | SNTG2 |
| 2q | | | | | | |
| | RP11-351E10 | 242375991-242565755 | 189764 | AC134880 | NEU4 | NEU4 |
| Size: 1 076 262 bp | RP11-875C22 | 242147376-242359508 | 212132 | AQ800748 | D2S2142 | ATG4B |
| | RP11-90E11 | 242009527-242187120 | 177593 | AQ281512 | RH47042 | BOK |
| Telo dist: 385 394 bp | RP11-952H3 | 241827539-242014017 | 186478 | AQ600636 | D2S1880 | SEPT2 |
| | RP11-185C8 | 241680132-241864592 | 184460 | AQ418167 | D2S1499E | PASK |
| | RP11-143H14 | 241489493-241648076 | 158583 | AQ373316 | RH36299 | |
| 3p | | | | | | |
| | RP11-306H5 | 159320-343406 | 184086 | AC026187 | D3S2359 | CHL1 |
| | RP11-114K9 | 333634-518315 | 184681 | AC011609 | D3S3938 | CHL1 |
| Size: 1 403 495 bp | RP11-775C23 | 576763-773499 | 196736 | AC087431 | AFM240XF12 | |
| | RP11-86C13 | 768337-958276 | 189939 | AC090044 | D3S1762 | |
| Telo dist: 159 320 bp | RP11-242C8 | 955503-1107788 | 152285 | AC087430 | SHGC-132794 | |
| | RP11-392M7 | 1064434-1255997 | 191563 | AC027123 | SHGC-82415 | CNTN6 |
| | RP11-416N8 | 1244099-1420317 | 176218 | AC034192 | SHGC-8915 | CNTN6 |
| | RP11-717M12 | 1388171-1562815 | 174644 | AC026214 | SHGC-111745 | CNTN6 |
| 3q | | | | | | |
| | RP11-159K3 | 199038951-199230435 | 191484 | AQ375640 | RH74944 | LMLN |
| | CTD-2529O9 | 198655574-198826526 | 170952 | AQ353056 | RH78729 | BDH1 |
| | RP11-114F20 | 198339452-198528164 | 188712 | AQ342618 | SHGC-37072 | DLG1 |
| | RP11-183C22 | 198146622-198339454 | 192832 | AQ416118 | WI-6636 | MFI2 |
| Size: 1 918 615 bp | RP11-927L5 | 197896915-198070611 | 173696 | AQ623198 | D3S2348 | PAK2 |
| | RP11-200I19 | 197700433-197837659 | 137226 | AC092933 | RH58112 | WDR53 |
| Telo dist: 271 392 bp | RP11-106N22 | 197589202-197656469 | 67267 | AC083822 | D3S2306 | UBXD7 |
| | RP11-447L10 | 197402231-197591199 | 188968 | AC069257 | RH58488 | PCYT1A |
| | RP11-185G19 | 197311820-197404234 | 92414 | AC139666 | RH17509 | |
| 4p | | | | | | |
| | RP11-1081C15 | 13419-238368 | 224949 | AC140865 | D4S3358 | ZNF718 |
| Size: 1 488 449 bp | CTD-2125B17 | 155621-281984 | 126363 | AQ668149 | D4S1655 | |
| | RP11-349C22 | 248197-418202 | 170006 | AQ543334 | D4S134 | ZNF141 |
| | RP11-71F5 | 358141-531029 | 172888 | AQ236993 | D4S134 | PIGG |
| | RP11-784E7 | 459895-620780 | 160886 | AQ520229 | D4S764 | PIGG |
| Telo dist: 13 419 bp | RP11-922G10 | 605835-783700 | 177865 | AQ679597 | D4S913 | MYL5 |
| | RP11-833E16 | 803840-1029391 | 225551 | AQ524142 | RH27153 | FGFRL1 |
| | RP11-728M1 | 983672-1163266 | 179594 | AQ513815 | D4S992 | FGFRL1 |
| | RP11-939A10 | 1103084-1277545 | 174462 | AQ564122 | D4S2552E | CTBP1 |
| | CTD-2547M24 | 1287251-1501868 | 214618 | AQ394261 | RH59237 | MAEA |
| 4q | | | | | | |
| | RP11-45F23 | 190520006-190668423 | 148417 | AC020698 | D4S1369 | |
| Size: 1 114 662 bp | RP11-256N12 | 190366130-190552194 | 186064 | AQ484851 | D4S592 | |
| | RP11-463B4 | 190179198-190387639 | 208441 | AQ635346 | D4S592 | |
| Telo dist: 604 640 bp | RP11-354H17 | 189958830-190151921 | 193091 | AC017063 | D4S1086 | |
| | RP11-1077O22 | 189778946-189960823 | 181877 | AQ744110 | RH46856 | |
| | RP11-636B14 | 189553761-189739148 | 185387 | AQ436326 | D4S187 | |
| 5p | | | | | | |
| | RP11-1006P13 | 387034-601876 | 301500 | AC113363 | D5S1924E | SDHA |
| Size: 1 578 368 bp | RP11-846K3 | 601882-783339 | 415115 | AC145151 | RH91398 | EXOC3 |
| | RP11-947G18 | 773300-977133 | 203834 | AQ564519 | D5S2254E | TRIP13 |
| | RP11-260H6 | 791202-1072995 | 281793 | AQ481879 | D5S2254E | BRD9 |
| Telo dist: 387034 bp | RP11-1040C13 | 1024509-1248692 | 224183 | AQ836977 | RH11663 | NKD2 |
| | RP11-117B23 | 1260045-1422139 | 162095 | AQ349447 | D5S2681 | TERT |
| | RP11-94J21 | 1377471-1540909 | 163438 | AQ281737 | D5S2681 | SLC6A3 |
| | RP11-464H7 | 1795659-1965402 | 169744 | AQ586375 | SHGC-153380 | IRX4 |
| 5q | | | | | | |
| | RP11-69N15 | 180429200-180616147 | 186947 | AQ265515 | SHGC-147769 | TRIM7 |
| Size: 1 149 052 bp | CTD-2530D22 | 180124020-180396206 | 272193 | AQ308731 | D5S2724 | BTLN8 |
| | RP11-451H23 | 179870132-180060155 | 190023 | AC122714 | D5S408 | FLT4 |
| Telo dist: 241 719 bp | RP11-282I19 | 179629463-179805650 | 176187 | AQ503682 | SHGC-148059 | GFPT2 |
| | RP11-252I14 | 179467095-179662550 | 195455 | AZ517447 | RH48160 | MAPK9 |
| 6p | | | | | | |
| | RP11-164H16 | 352885-522691 | 169806 | AQ380798 | RH63985 | EXOC2 |
| Size: 910 753 bp | RP11-764L8 | 450983-623958 | 172975 | AQ620817 | RH45976 | EXOC2 |
| | RP11-1104N17 | 646645-856285 | 209640 | AQ678197 | RH37410 | |
| Telo dist: 352 885 bp | CTD-2509I12 | 826242-1013569 | 187327 | AQ265243 | RH37410 | |
| | RP11-939G22 | 924064-1099477 | 175413 | AQ564164 | RH37407 | |
| | CTD-2537K7 | 1082250-1263638 | 181388 | AQ352875 | RH37403 | |
| 6q | | | | | | |
| | RP11-614P3 | 169746523-169946788 | 200265 | AQ361238 | RH60746 | PHF10 |
| Size: 1 010 693 bp | RP11-369H17 | 169584276-169746513 | 162237 | AQ530605 | RH46814 | WDR27 |
| | RP11-135E20 | 169430897-169619248 | 188351 | AQ380078 | RH45974 | WDR27 |
| Telo dist: 953 204 bp | RP11-417E7 | 169221863-169333040 | 111177 | AL136129 | SHGC-57468 | |
| | RP11-35J6 | 168936095-169093666 | 157571 | AL513210 | SHGC-104666 | |
| 7p | | | | | | |
| | RP13-689E11 | 477 568-639 706 | 162 138 | AC147651 | D7S2794 | PDGFA |
| | CTD-3028G18 | 677437-900304 | 222867 | AQ094966 | D7S2644 | UNC84A |
| Size: 1 185 039 bp | CTD-3168K8 | 889961-1058173 | 168212 | AQ784510 | RH14754 | CENTA1 |
| | RP11-1133D5 | 1101944-1264436 | 162492 | AQ721269 | RH65680 | ZFAND2A |
| Telo dist: 477 568 bp | RP11-1080I13 | 1256438-1459423 | 202985 | AQ743976 | RH46485 | MICAL-L2 |
| | CTD-3226F4 | 1435253-1662607 | 227354 | AQ205035 | RH26298 | MAFK |
| 7q | | | | | | |
| | RP11-1112M14 | 158602180-158788150 | 185970 | AQ747375 | RH48601 | VIPR2 |
| Size: 1 238 519 bp | RP11-664B5 | 158404470-158595970 | 191500 | AQ611285 | D7S749 | VIPR2 |
| | CTD-2554I6 | 158081863-158344153 | 262298 | AQ390675 | D7S2310 | LUZP5 |
| Telo dist: 33 274 bp | CTD-2033H20 | 157802288-158058234 | 255946 | AQ230709 | D7S467 | PTPTRN2 |
| | RP11-11B21 | 157752948-157964627 | 211679 | AC019043 | D7S467 | PTPTRN2 |
| | RP11-518I12 | 157549631-157754947 | 205316 | AC093856 | D7S1476 | PTPTRN2 |
| 8p | | | | | | |
| | RP11-1072H3 | 476255-658396 | 182141 | AQ680374 | RH65733 | ERICH1 |
| Size: 1 168 861 bp | RP11-166P22 | 686332-863608 | 177276 | AQ381144 | D8S462 | |
| | RP11-973L9 | 865361-1032010 | 166649 | AQ669172 | SHGC-149177 | |
| Telo dist: 476 255 bp | CTD-2613K14 | 1070949-1206360 | 135411 | AC124318 | NONE | |
| | CTD-2523L18 | 1257226-1466501 | 209275 | AQ279685 | WI-1986 | DLGAP2 |
| | RP11-666I19 | 1480818-1645116 | 164298 | AQ518141 | SHGC-172453 | DLGAP2 |
| 8q | | | | | | |
| | RP11-590B21 | 145963321-146131249 | 167928 | AQ791630 | D8S595 | ZNF7 |
| Size: 1 128 540 bp | RP11-1143I12 | 145825257-145978282 | 153025 | AC134685 | SHGC-172477 | |
| | RP11-620H1 | 145536611-145740218 | 203607 | AQ401678 | RH104428 | KIFC2 |
| Telo dist: 143 577 bp | CTD-3232M19 | 145403397-145494495 | 91098 | AC110280 | WI-17683 | BOP1 |
| | CTD-3065J16 | 145002709-145218050 | 215341 | AC109322 | RH80294 | PLEC1 |
| 9p | | | | | | |
| | RP11-996P21 | 236699-418421 | 181722 | AQ703451 | RH40430 | DOCK8 |
| Size: 1 062 400 bp | RP11-675G5 | 442110-642967 | 200857 | AQ455361 | D9S989E | ANKRD15 |
| | RP11-696A8 | 656340-859501 | 203161 | AQ431231 | RH47783 | DMRT1 |
| Telo dist: 236 699 bp | RP11-960F12 | 913347-1111094 | 197747 | AQ743131 | D9S999E | DMRT3 |
| | CTD-2537D15 | 1078037-1299099 | 221062 | AQ356214 | WI-2625 | — |
| 9q | | | | | | |
| | RP11-937L7 | 139785404-139993460 | 208056 | AQ564902 | WI-3929 | EHMT1 |
| | RP11-47N19 | 139634568-139774734 | 140167 | AQ202821 | RH40407 | EHMT1 |
| Size: 2 072 351 bp | RP11-417A4 | 139523178-139716008 | 192830 | AL161451 | RH40445 | GRIN1 |
| | RP11-48C7 | 139459573-139634567 | 174994 | AL365502 | RH62727 | NELF |
| Telo dist: 279 792 bp | CTD-2377P2 | 139297032-139429236 | 132220 | AQ116453 | RH47760 | |
| | RP11-350O14 | 139029415-139220879 | 191464 | AQ630674 | RH40445 | ENTPD2 |
| | RP11-673E5 | 138946390-139158292 | 211902 | AQ409597 | RH40414 | ABCA2 |
| | RP11-769N4 | 138736552-138946296 | 209744 | AQ497228 | RH76770 | EDF1 |
| | CTD-2551F21 | 138554306-138754280 | 199974 | AQ391590 | RH62709 | EGFL7 |
| | RP11-413M3 | 138370650-138559111 | 188462 | AL592301 | RH40348 | CARD9 |
| | RP11-83N9 | 138121792-138276710 | 154918 | AL138781 | SHGC-154953 | LHX3 |
| | RP11-662J2 | 137921109-138121782 | 200674 | AQ518791 | SHGC-147426 | BTBD14A |
| 10p | | | | | | |
| | RP11-567B24 | 99677-279309 | 179632 | BH140890 | D10S1212 | ZMIND11 |
| Size: 1 163 581 bp | RP11-486H9 | 286973-466727 | 179754 | AL603831 | RH51265 | BS69 |
| | RP11-1077M19 | 461663-638728 | 177065 | AQ743812 | SHGC-153467 | DIP2C |
| Telo dist: 99 677 bp | RP11-349B19 | 642617-855859 | 213242 | AQ527417 | WI-9969 | DIP2C |
| | RP11-259N11 | 908097-1060260 | 152163 | AQ482971 | RH40165 | LARP5 |
| | RP11-354A11 | 1037000-1263258 | 226258 | AQ530632 | RH79159 | WDR37 |
| 10q | | | | | | |
| | RP11-108K14 | 135078861-135240498 | 161637 | AQ323968 | D10S2490 | CYP2E1 |
| Size: 1 149 860 bp | RP11-122K13 | 134955982-135072292 | 116310 | AL360181 | D10S1711 | TUBGCP2 |
| | RP11-1022E21 | 134703784-134906603 | 202819 | AQ702693 | RH51132 | |
| Telo dist: 134 239 bp | RP11-97M24 | 134607245-134778110 | 170865 | AQ318170 | NONE | |
| | RP11-288G11 | 134340940-134549003 | 208063 | AL392043 | D10S2346 | INPP5A |
| | RP11-500B2 | 134228392-134341039 | 112647 | AL356603 | D10S212 | INPP5A |
| | RP11-384O10 | 134090638-134276997 | 186359 | AQ534302 | D10S316 | INPP5A |
| 11p | | | | | | |
| | RP11-326C3 | 205457-346450 | 140993 | AC136475 | D11S4895 | IFITM3 |
| Size: 2 438 350 bp | RP11-1007G14 | 368238-557259 | 189021 | AC142165 | RH99331 | DEAF1 |
| | RP11-754B17 | 585784-740976 | 155192 | AC131934 | RH14840 | DEAF1 |
| | RP11-51L17 | 764448-942590 | 178142 | AQ052364 | RH47994 | LRDD |
| | RP11-613G2 | 942562-1118625 | 176063 | AQ368555 | D11S3523 | AP2A2 |
| | CTD-3092J9 | 1089844-1286357 | 196513 | AS128188 | RH46115 | TOLLIP |
| Telo dist: 205 457 bp | RP11-89F15 | 1299307-1482389 | 183083 | AQ284680 | SHGC-146355 | BRSK2 |
| | RP11-1059M21 | 1482390-1689774 | 207384 | AQ832214 | D11S3570 | DUSP8 |
| | RP11-373H8 | 2261657-2468447 | 206790 | AQ531134 | RH79705 | TSPAN32 |
| | RP11-1023M19 | 2468462-2643807 | 175341 | AQ595456 | D11S4726 | KCNQ1 |
| 11q | | | | | | |
| | RP11-410I24 | 133413709-134301418 | 887709 | AC034162 | D11S1110 | THYN1 |
| Size: 1 129 412 bp | RP11-469N6 | 133983990-134156497 | 172507 | AP001999 | D11S1185 | |
| | RP11-627G23 | 133832598-133898497 | 65899 | AP004608 | SHGC-148671 | |
| Telo dist: 150 966 bp | RP11-164K8 | 133527689-133732868 | 205179 | AQ378986 | D11S4071 | ACAD8 |
| | RP11-368H5 | 133348982-133518619 | 169637 | AQ528884 | D11S3481 | JAM3 |
| | RP11-259H11 | 133172006-133349688 | 177682 | AQ482781 | D11S4457 | SPATA19 |
| 12p | | | | | | |
| | RP11-722I13 | 138957-345401 | 206444 | AQ462206 | D12S2046 | SLC6A13 |
| | RP11-55P12 | 340245-484313 | 144068 | AQ115910 | SHGC-58503 | JARID1A |
| | CTD-2535C18 | 498465-709998 | 211533 | AQ351686 | D12S522 | NINJ2 |
| | RP11-388A16 | 710002-860797 | 150795 | AC004765 | D12S1455 | WNK1 |
| Size: 1 652 608 bp | RP11-359B12 | 858584-1012956 | 154372 | AC004803 | D12S1913 | RAD52 |
| | RP11-159N18 | 1105281-1268766 | 163485 | AQ374122 | RH48779 | RAB6IP2 |
| Telo dist: 138 957 bp | RP11-714I24 | 1266682-1474863 | 208181 | AQ438448 | D12S2047 | RAB6IP2 |
| | RP11-73H11 | 1413244-1555892 | 142648 | AQ266984 | D12S939 | FBXL14 |
| | RP11-783G21 | 1593777-1791565 | 197788 | AQ458430 | RH66215 | WNT5B |
| 12q | | | | | | |
| | CTD-2140B24 | 132128913-132289534 | 160621 | AC026786 | D12S399 | ZNF140 |
| Size: 1 413 614 bp | RP11-386I8 | 132034460-132178738 | 144278 | AC073911 | D12S399 | ZNF26 |
| | RP11-46H11 | 131855460-132036476 | 181016 | AC127070 | RH98685 | CHFR |
| Telo dist: 60 000 bp | CTD-3237H24 | 131516349-131759664 | 243315 | AQ209297 | RH16876 | POLE |
| | RP11-867C16 | 131361271-131545666 | 184395 | AQ818926 | SHGC-146686 | |
| | CTD-2519K8 | 131054254-131272060 | 217806 | AQ277627 | RH39825 | DDX51 |
| | CTD-2522P9 | 130875920-131078105 | 202192 | AQ280680 | RH91194 | PUSI1 |
| 13q | | | | | | |
| | RP11-569D9 | 113930807-114103243 | 172436 | AL160396 | RH10582 | CDC16 |
| Size: 1 325 533 bp | RP11-245B11 | 113770458-113932864 | 162406 | AL161774 | RH104222 | RASA3 |
| | RP11-199F6 | 113473995-113644014 | 170019 | BX072579 | D13S1513 | GAS6 |
| Telo dist: 39 737 bp | RP11-230F18 | 113166605-113351493 | 184888 | AL442125 | RH53220 | TMCO3 |
| | RP11-391H12 | 112999602-113025743 | 26141 | AL136221 | WI-9160 | LAMP1 |
| | RP11-98F14 | 112777710-112939870 | 162160 | AQ319320 | RH73716 | F7 |
| 14q | | | | | | |
| Size: 1 306 717 bp | RP11-417P24 | 105267349-105437150 | 169801 | AL122127 | RH18069 | IGH |
| | RP11-731F5 | 105018846-105195083 | 176237 | AL928742 | RH68957 | TMEM121 |
| Telo dist: 931 435 bp | RP11-435F10 | 104781176-104980676 | 199500 | AZ081951 | RH44444 | BRF1 |
| without 158A2 | RP11-1087P8 | 104677732-104876215 | 198483 | AL512355 | RH53818 | JAG2 |
| | RP11-44N21 | 104552642-104711108 | 158466 | AL512356 | H14A1767 | JAG2 |
| | RP11-18C13 | 104381504-104533148 | 151644 | B88076 | RH53647 | PDL4 |
| | RP11-982M15 | 104130433-104336369 | 205936 | AL583722 | RH75756 | AKT1 |
| 15q | | | | | | |
| | RP11-829L22 | 99860523-100046963 | 186440 | AQ814024 | RH25483 | TM2D3 |
| Size: 1 017 092 bp | RP11-530H6 | 99746892-99914703 | 167811 | AZ301327 | RH44112 | PCSK6 |
| | RP11-299F21 | 99550365-99734009 | 183644 | AQ504845 | D15S910 | CHSY1 |
| Telo dist: 291 952 bp | CTD-2502E10 | 99316896-99544556 | 227660 | AQ260632 | RH44810 | LLRK1 |
| | CTD-3211D11 | 99029871-99262670 | 232799 | AQ184057 | RH48812 | ALDH1A3 |
| 16p | | | | | | |
| | CTD-3077J14 | 16715-199854 | 183139 | AQ122076 | RH71405 | C16orf35 |
| Size: 1 336 539 bp | RP11-26D18 | 229696-414158 | 184462 | B86834 | RH1388 | AX1N1 |
| | RP11-598I20 | 273376-466227 | 192851 | AQ339547 | RH70775 | MRPL28 |
| Telo dist: 16 715 bp | CTD-2524J22 | 528660-729508 | 200848 | AQ310739 | RH66982 | FBXL16 |
| | RP11-252I11 | 865543-958181 | 92638 | AC046157 | NONE | |
| | CTD-3158F9 | 976506-1171082 | 194576 | AQ803127 | RH54588 | SOX8 |
| | CTD-2503P16 | 1155596-1353254 | 197658 | AQ275751 | RH70600 | UBE2I |
| 16q | | | | | | |
| | RP11-417N6 | 88466848-88643471 | 176623 | AZ081868 | RH54513 | GAS8 |
| Size: 1 082 241 bp | RP11-7D23 | 88297302-88297857 | 169 540 | AZ519973 | SHGC 32044 | |
| | RP11-1089G18 | 88081018-88285940 | 204922 | AQ696957 | SHGC-152531 | SPG7 |
| Telo dist: 183 783 bp | RP11-880I20 | 87847056-68067286 | 220230 | AQ833570 | RH69658 | ANKRD11 |
| | RP11-1122C1 | 87721624-87875044 | 153420 | AQ719790 | SHGC-149631 | CDH15 |
| | RP11-933C23 | 87561230-87724152 | 162922 | AC136911 | SHGC-145880 | CBFA2T3 |
| 17p | | | | | | |
| | RP11-755K24 | 503724-687822 | 184098 | AQ620192 | D17S1569 | VPS53 |
| | RP11-356I18 | 707755-880135 | 172380 | AQ535844 | WI-7295 | NXN |
| Size: 1 573 433 bp | RP11-934E17 | 791217-980108 | 188891 | AQ691291 | WI-7295 | ABR |
| | CTD-2507J6 | 964370-1044519 | 80149 | AC107911 | RH46166 | ABR |
| Telo dist: 503 724 bp | RP11-818O24 | 1099692-1299375 | 199683 | AC032044 | D17S1582 | YWHAE |
| | RP11-961A15 | 1400535-1617783 | 217248 | AC130343 | RH32330 | PRPF8 |
| | RP11-968I3 | 1035598-1766861 | 731263 | AC132811 | RH14944 | SKIP |
| | CTD-2545H1 | 1715136-1881258 | 166122 | AC099684 | RH45618 | RPA1 |
| | RP11-667K14 | 1872529-2077157 | 204628 | AC090617 | RH36098 | SMG6 |
| 17q | | | | | | |
| | CTD-2519K10 | 78335862-78488483 | 457855 | AQ277631 | SHGC-147547 | TBCD |
| | RP11-388C12 | 78200000-78311473 | 111473 | AC024361 | D17S724 | RAB40B |
| Size: 1 494 456 bp | RP11-525L23 | 77424566-78592153 | 1167587 | AC023786 | RH11500 | TBCD |
| | RP11-1087N2 | 77771385-78520665 | 749280 | AC137090 | D17S1282 | TBCD |
| Telo dist: 286 259 bp | RP11-51H16 | 77524868-77690741 | 165873 | AQ053634 | D17S704 | ASPSCR1 |
| | RP11-634L10 | 77390102-77562577 | 172475 | AQ441944 | RH65274 | SIRT7 |
| | RP11-765O14 | 76994027-77189725 | 195698 | AQ514170 | WI-7522 | ACTG1 |
| 18p | | | | | | |
| | RP11-683L23 | 5982-142961 | 136979 | AP001005 | RH65142 | ROCK1 |
| Size: 1 098 419 bp | RP11-76H24 | 203477-370531 | 167054 | AQ285541 | D18S551 | COLEC12 |
| | RP11-133D9 | 370566-543299 | 172733 | AQ349704 | D18S92 | COLEC12 |
| Telo dist: 5 982 bp | CTD-2593J12 | 591322-771970 | 180648 | AQ473690 | RH11010 | TYMS |
| | RP11-1152E8 | 729531-906049 | 176518 | AQ749285 | D18S1078 | YES1 |
| | RP11-1005B18 | 908890-1104401 | 195511 | AQ670912 | D18S1077 | |
| 18q | | | | | | |
| | RP11-565D23 | 75936873-76103217 | 166344 | AC068530 | D18S497 | PARD6G |
| Size: 1 182 640 bp | RP11-93F7 | 75790939-75945751 | 154812 | AQ323706 | RH1664 | TXLN4A |
| | CTD-3110E17 | 75603460-75757760 | 154300 | AQ152063 | D18S1015 | CTDP1 |
| Telo dist: 13 936 bp | RP11-803N2 | 75432037-75603441 | 171404 | AQ525286 | RH55591 | CTDP1 |
| | RP11-196B3 | 75261845-75424109 | 162264 | AC018445 | RH1999 | NFATC1 |
| | RP11-841P22 | 75089275-75328424 | 239149 | AQ826661 | WI-11906 | ATP9B |
| | RP11-767J19 | 74920577-75083631 | 163054 | AQ458007 | RH9976 | ATP9B |
| 19p1 | | | | | | |
| | RP11-575H1 | 209326-374094 | 164 781 | AZ301204 | SHGC-146248 | THEG |
| | CTD-2577I7 | 400897-520334 | 119437 | AQ427448 | RH55732 | MADCAM1 |
| Size: 2 534 244 bp | CTD-2589F14 | 429636-653131 | 223495 | AQ376407 | D19S814 | HCN2 |
| | CTD-2378A10 | 609535-815726 | 206191 | AQ109918 | RH75761 | PALM |
| Telo dist: 209 326 bp | RP11-75H6 | 902642-1095485 | 192843 | AQ266693 | RH25429 | ABCA7 |
| | CTD-3193P3 | 1095512-1152241 | 56729 | AQ790060 | WI-17215 | KIAA0963 |
| | RP11-317H11 | 1331125-1495933 | 164808 | AQ542734 | RH27114 | DAZAP1 |
| | RP11-846C18 | 1756204-1947303 | 191099 | AQ799643 | RH11579 | SCAMP4 |
| | RP11-660021 | 2003350-2184507 | 181157 | AQ434085 | RH10739 | AP3D1 |
| | CTD-3036J17 | 2511770-2743570 | 231800 | AQ096988 | D19S1028 | GNG7 |
| 19q | | | | | | |
| | CTD-2575K21 | 63638468-63770533 | 132065 | AQ423051 | RH32622 | ZNF324 |
| | RP11-357E24 | 63445258-63638426 | 193168 | AQ540962 | RH55926 | ZNF8 |
| Size: 1 030 032 bp | RP11-706G10 | 63385977-63711682 | 325705 | AC023149 | RH55939 | A1BG |
| | RP11-91H11 | 63223240-63387841 | 164601 | AQ283401 | RH79637 | ZNF447 |
| Telo dist: 41 118 bp | CTD-2583A14 | 63048608-63182459 | 133851 | AC010326 | RH98684 | ZNF587 |
| | RP11-1069H17 | 62854921-63042914 | 187993 | AQ664338 | RH98527 | ZNF154 |
| | RP11-103G13 | 62608394-62794238 | 185844 | AQ323224 | RH32542 | ZNF550 |
| 20p | | | | | | |
| | CTD-2506E2 | 37211-226388 | 189177 | AQ262614 | D20S210 | DEFB126 |
| Size: 1 609 168 bp | RP11-300H9 | 239394-411347 | 171953 | AQ508904 | RH17493 | SOX12 |
| | RP11-701C1 | 376497-543442 | 166945 | AQ408296 | RH511842 | CSNK2A1 |
| Telo dist: 37 211 bp | RP11-60D10 | 549244-734702 | 185458 | AQ198631 | RH32985 | SCRT2 |
| | RP11-978M13 | 760084-936972 | 176888 | AQ606367 | RH67480 | ANGPT4 |
| | RP11-153M12 | 893802-1085086 | 191284 | AQ387838 | WI-1352 | RSPO4 |
| | RP11-268D6 | 1015548-1173495 | 157947 | AQ478112 | RH75207 | PSMF1 |
| | RP11-43K22 | 1145645-1318481 | 172837 | AQ199569 | SHGC-32807 | SNPH |
| | RP11-621G24 | 1275633-1452295 | 176663 | AQ401933 | SHGC-32807 | FKBP1A |
| | RP11-974O11 | 1437272-1646379 | 209108 | AQ753972 | SHGC-56794 | SIRPD |
| 20q | | | | | | |
| | CTD-2022N21 | 62215516-62317284 | 101768 | AQ228698 | STSG24299 | MYT1 |
| | CTD-2559F2 | 61924110-62120608 | 196 498 | AQ424201 | RH91387 | TPD52L2 |
| | CTD-3231H17 | 61807905-61974060 | 166155 | AQ192102 | RH98655 | BTBD4 |
| Size: 1 474 6117 bp | RP11-95N13 | 61564623-61727698 | 163075 | AQ315674 | RH78083 | EEF1A2 |
| | RP11-261N11 | 61332296-61516517 | 184221 | AL121827 | RH12050 | ARFGAP1 |
| Telo dist: 118 680 bp | CTD-3051D12 | 61038964-61249909 | 210945 | AQ134307 | RH67463 | BC038593 |
| | RP11-477N6 | 60842673-61032192 | 189519 | AQ637343 | RH64147 | DIDO1 |
| 21q | | | | | | |
| | RP11-1000I21 | 46743970-46940213 | 196243 | AQ714296 | RH66875 | DIP2A |
| Size: 1 101 746 bp | CTD-3217A14 | 46620980-46833176 | 212196 | AQ181558 | D21S1272 | PCNT |
| | RP11-34P17 | 46391180-46582695 | 191515 | AQ045126 | RH12647 | MCM3AP |
| Telo dist: 4 110 bp | RP11-640F21 | 46144350-46311763 | 167413 | AQ411047 | D21S403 | COL6A1 |
| | RP11-892E8 | 46068771-46280397 | 211626 | AQ623937 | D21S403 | BCBP3 |
| | RP11-93F5 | 45979578-46151974 | 172396 | AQ323704 | D21S1574 | BCBP3 |
| | RP11-48G23 | 45838467-46022220 | 183753 | AQ202789 | D21S402 | |
| 22q | | | | | | |
| | RP11-825H3 | 49161956-49345964 | 184008 | AQ793726 | RH46917 | SBF1 |
| Size: 827 167 bp | CTD-2579L10 | 48886805-49061533 | 174728 | AQ475194 | RH57822 | TUBGCP6 |
| Telo dist: 345 468 bp | RP11-931F19 | 48767143-48969501 | 202358 | AQ565223 | D22S1056 | MOV10L1 |
| | RP11-49N13 | 48518797-48673310 | 154519 | AQ051769 | D22S1052 | BRD1 |
| XpYp | | | | | | |
| | RP11-91D5 | 272751-407801 | 135050 | AQ283277 | DXYS60 | |
| Size: 1 639 430 bp | RP11-800K15 | 433105-614235 | 181130 | AQ523944 | DXYS28 | SHOX |
| | RP11-946P8 | 643506-870272 | 226766 | AQ600003 | DXYS6796 | |
| | RP11-892B14 | 1108980-1329314 | 220334 | AQ819426 | G66030 | CSF2RA |
| Telo dist: 272 751 bp | RP11-261P4 | 1457956-1620348 | 162392 | AL683870 | DXYS140 | ASMTL |
| | CTD-3047L21 | 1610184-1762933 | 152749 | AQ134439 | DXYS155E | ASMT |
| | CTD-3239E12 | 1685500-1912181 | 226682 | AQ211694 | DXYS155E | ASMT |
| XqYq | | | | | | |
| | RP11-479B17 | 154703321-154862054 | 158733 | AQ629322 | DXS7859 | SYBL1 |
| | RP11-175B5 | 154533822-154703245 | 169423 | AQ418259 | G65952 | SPRY3 |
| | RP11-954J6 | 154388631-154559759 | 171128 | AQ624034 | DXS1108 | TMLHE |
| Size: 1 426 929 bp | RP11-405N23 | 154111989-154275960 | 163971 | AZ301102 | RH70015 | CLIC2 |
| | RP11-207O16 | 153896624-154041384 | 144760 | AQ415867 | STSG604444 | CXorf53 |
| Telo dist: 51 700 bp | RP11-524G17 | 153688415-153923534 | 235119 | AL645722 | STSG604337 | F8 |
| | RP11-103M23 | 153435125-153609357 | 174232 | AQ611327 | STSG604213 | IKBKG |

### 1.1. BAC Culture

The BACs are ordered from the Centre National de Séquençage [National Sequencing Center] in Evry (Génoscope), which supplies them in the form of colonies in Petri dishes.

Each BAC is cultured according to the following protocol: 500 μl of TB culture medium containing chloramphenicol (25 m/ml) is deposited in each of the wells of the 96-well plate. One colony of each bacteria clone is then cultured per well. The plates are incubated for 24 hours at 37° C. with agitation. The cultures are stopped by adding 500 μl of TB medium/30% glycerol. This will form the glycerol stock plate, from which 3 subcultures will be generated: the S (stock) plate, the B (backup) plate and the W (working) plate, each containing 150 μl of culture. The W plate is used to obtain the DNA. The plates are stored at −80° C. in different freezers.

### 1.2. DNA Amplification by RCA (Rolling Circle Amplification):

This is the DNA production step

### a) Principle

DNA amplification by RCA is based on the replication, at a constant temperature, of short single-stranded circular DNA segments. The reaction consists of the extension of hexameric primers randomly bound to the DNA template, making it possible to obtain copies of single-stranded linear DNA, which can in turn be used as a template for the hybridization of other primers. This makes it possible to avoid the conventional steps of phenol-chloroform extraction.

### b) Protocol: GE Healthcare Europe Kit: Ref 25-6400-80

This step uses the protocol provided with the RCA kit. It is carried out in a 96-well plate, which allows the simultaneous production of DNA (10 μg) from 4 μl of culture of 96 different clones.

A final DNA concentration of 100 ng/μl (10 μg in 100 μl of water) is obtained. The amplification is verified on an agarose gel.

### 1.3. Labeling

This is the step that will enable fluorescent molecules to be incorporated into the DNA.

Each RCA product is labeled by means of the nick-translation principle.

A solution containing 20 ng/μl of DNA labeled with a fluorochrome is obtained in 2 h 30.

The labelings are verified as follows:

4 ml of the labeling reaction are deposited on a 1% agarose gel in order to visualize, under UV, the DNA into which the fluorochromes have been integrated. This makes it possible to assess the quality of the labeling.

1.4. Probe Precipitation This step allows the production of the ready-to-use probe.

200 ng of each of the probes are precipitated in the presence of 50× (i.e. 10 μg) Cot1 DNA (DNA rich in repeat sequences which will block the repeat sequences existing in the probe), with 0.15M of 3M NaCl and 3 volumes of 100% ethanol. The pellet is then taken up in 6 μl of hybridization buffer (50% formamide, 2×SSC, 0.1% SDS, 40 mM NaH₂PO₄/NaHPO₄). The concentration at which the probe is used is therefore approximately 30 ng/μl.

### 1.5. Verification of Probe Localization by FISH

Before constituting a probe contig, each probe (BAC) was verified by FISH on metaphase. All the BACs generating a signal on several chromosomes or not in place on the expected chromosome, were replaced.

### 1.6. Formation of the “Pool”

This makes it possible to considerably simplify the process by handling only 41 DNA pools in place of the 286 clones constituting the latter.

The “pool” plate is formed from the working plate. For each chromosomal end, 20 μl of TB/glycerol, of each bacterial clone corresponding to a BAC constituting the contig, are taken and then mixed in a single well of a 96-well plate. A plate is thus formed which contains 41 mixtures of bacterial clones from which probes specific for each of the 41 chromosomal ends are produced very rapidly, according to the methods previously described (culture, RCA, labeling, and verification by FISH).

### Starting from the W Plate

### 20 μl of Each BAC

| | | | | | | | | | | | |
|---|---|---|---|---|---|---|---|---|---|---|---|
| 1p | 1q | 2p | 2q | 3p | 3q | 4p | 4q | 5p | 5q | 6p | 6q |
| 7p | 7q | 8p | 8q | 9p | 9q | 10p | 10q | 11p | 11q | 12p | 12q |
| 13q | 14q | 15q | 16p | 16q | 17p | 17q | 18p | 18q | 19p | 19q | 20p |
| 20q | 21q | 22q | XYp | XYq | | | | | | | |

41 ends plate = “pool” plate

### Example 2

### Production of the MTP (“Mega Telomeric Probes”) System

The strategy adopted resulted in the production of 41 probes which are labeled by means of a combination of 5 fluorochromes: FITC, rhodamine, DEAC, biotin (revealed with streptavidin-Cy5) and digoxigenin (revealed with Cy5.5).

The ends of the same chromosome are labeled with the same color combination.

### 2.1 Labeling of the Two Groups

Each group was constructed in such a way as to avoid bringing together chromosomes resembling one another from a cytogenetic point of view (21 and 22, in two different groups, for example).

All the ends which contain the same fluorochrome are labeled together by nick-translation and according to tables 3A and 3B below. For group I, 1960 ng of DNA were labeled with FITC, 2120 ng with rhodamine, 1050 ng with coumarin, 1983 ng with biotin and 1860 ng with digoxigenin, in respective volumes of 83, 98, 50, 88 and 84 μl. For group II, 1810 ng were labeled with FITC, 1860 ng with rhodamine, 1607 ng with biotin and 1944 ng with digoxigenin. This difference in amount of DNA labeled is intended to reinforce the signal of certain weaker ends.

Tables 3A and 3B: Tables of Labeling of the Two Groups with Combinatorial Table

A-

| GROUP I | FITC | Rhoda | Couma | Cy5 | Cy5.5 |
|---|---|---|---|---|---|
| 1p/1q | green | | | yellow | |
| 2p/2q | green | | | | |
| 4p/4q | | | | yellow | fuschia |
| 6p/6q | green | red | | yellow | fuschia |
| 7p/7q | | | blue | | |
| 8p/8q | | red | | yellow | |
| 11p/11q | | | | yellow | |
| 13q | green | red | | yellow | |
| 15q | green | red | | | |
| 17p/17q | | red | | yellow | fuschia |
| 19p/19q | | red | | | |
| 21q | green | | | yellow | fuschia |

B-

| GROUP II | FITC | Rhoda | Cy5 | Cy5.5 |
|---|---|---|---|---|
| 3p/3q | green | red | | |
| 5p/5q | | | yellow | |
| 9p/9q | | red | | |
| 10p/10q | | red | yellow | fuschia |
| 12p/12q | green | | yellow | fuschia |
| 14q | green | red | yellow | fuschia |
| 16p/16q | green | | yellow | |
| 18p/18q | | red | yellow | |
| 20p/20q | | | yellow | fuschia |
| 22q | green | red | yellow | |
| XpYp/XqYq | green | | | |

### 2.2. Precipitation of the Groups

For a hybridization and per group, all the labeled probes are precipitated simultaneously and taken up in 6 μl of hybridization buffer.

### 2.3. Probe Hybridization

The probes referred to as “Mega telomeric probes” (MTPs) are hybridized according to the same protocol as for the BACs, but require slides with two deposits in order to simultaneously process the two groups of chromosomes.

### 2.4. Revealing of Slides and Analysis

The slides are washed in a bath of 1×SSC at 72° C. for 5 min, and then revealed with two layers of antibodies in order to reveal the probes labeled with biotin and digoxigenin: the first layer of antibodies (50 μl of 1× sodium bicarbonate+2 μl of anti-streptavidin Cy5 (1 mg/ml) for the biotin+0.5 μl of mouse anti-digoxigenin), and the second layer (50 μl of 1× sodium bicarbonate+1 μl of anti-mouse Cy5.5 (1 mg/ml)) makes it possible to reveal the digoxigenin.

The slides are mounted with 10 μl of a solution of DAPI/Vectashield® (Vector Laboratories) and then covered with a 24×60 coverslip.

The slides are observed using an epifluorescence microscope equipped with a camera and appropriate filters, and the metaphases are analyzed using an analyzing system.

### Example 3

### Demonstration of a Balanced Subtelomeric Translocation t(5; 1)(q35; p15)

In order to test the resolution of the probes, the inventors carried out a hybridization of said probes in a patient with acute myeloid leukemia associated with a cryptic telomeric abnormality, t(5; 11)(q35; p15), therefore invisible by conventional cytogenetics.

This translocation recombines the NUP98 gene encoding a 98 kDa nucleoporin located at 3 Mb from the telomere of the short arm of chromosome 11 and the NSD1 gene encoding a nuclear receptor SET domain protein located at 4 Mb from the telomere of the long arm of chromosome 5.

The resolution of the band karyotype is 5 to 10 Mb (often 10 Mb for the chromosomes of leukemic cells). The MTP system of the invention made it possible to easily detect this abnormality.

In order to confirm that it is indeed a translocation reorganizing the distal end of the long arm of a chromosome 5 and that of the short arm of a chromosome 11, the inventors hybridized the probes specific for the short and long arms of chromosome 11 (respectively labeled with coumarin (in blue) and with biotin revealed with streptavidin Cy5 (yellow) and those specific for the short (labeled with FITC, green) and the long (labeled with rhodamine, red) arms of chromosome 5. The inventors were thus able to visualize a t(5; 11)(qter; pter) translocation.

1. An in vitro method for detecting chromosomal abnormalities in an animal, comprising:
the in situ hybridization of n chromosomes from a metaphase chromosome preparation with sets of a plurality of nucleic acids, each set hybridizing, over a length of 700 000 to 3 000 000 contiguous bp, specifically to the subtelomeric ends specific to said chromosomes, each set being detectably labeled with a fluorochrome, such that each chromosome is distinguishable by a particular fluorochrome or a particular fluorochrome combination,
n being an integer between 2 and the total number of chromosomes of said animal, the total number of fluorochromes used being an integer less than n,
the detection of the fluorescence emitted by the fluorochromes, whereby chromosomal abnormalities are detected.

2. The method according to claim 1, the method being implemented for detecting at least twelve chromosomes.

3. The method according to claim 1, the method being implemented for all the chromosomes of the metaphase chromosome preparation.

4. The method according to claim 1, in which the sets of a plurality of nucleic acids are produced by amplification of one or more BACs into which one or more fragments of the subtelomeric end of said chromosome have been inserted.

5. The method according to claim 4, in which the amplification is a rolling circle amplification (RCA).

6. The method according to claim 1, in which the nucleic acids of at least one set are labeled with nucleotides conjugated with a molecule specifically recognized by a ligand labeled with a fluorochrome.

7. The method according to claim 1, in which the nucleic acids of at least one set are labeled with nucleotides conjugated with a fluorochrome.

8. The method according to claim 1, in which, for each chromosome, the subtelomeric ends of the short arm and of the long arm are labeled with the same fluorochrome or with the same fluorochrome combination.

9. The method according to claim 1, in which the animal is a human being.

10. The method according to claim 1, for the diagnosis of a constitutional chromosomal pathological condition.

11. The method according to claim 1, for the diagnosis of a cancer.
